(19) United States
(12) Patent Application Publication
(10) Pub. No.: US 2011/0301425 A1
(43) Pub. Date: Dec. 8, 2011

(54) Inductive Task Light For Surgical Console

(75) Inventors: Mikhail Boukhny, Laguna Niguel, CA (US); Christopher C. Jung, Mission Viego, CA (US); John Koontz, Corona, CA (US); David A. Thoe, Laguna Hills, CA (US); Asheesh Sah, Irvine, CA (US)

(73) Assignee: ALCON RESEARCH, LTD., Fort Worth, TX (US)

(21) Appl. No.: 12/793,079

(22) Filed: Jun. 3, 2010

(51) Int. Cl. A61B 1/06

(52) U.S. Cl. 600/249

(57) ABSTRACT

A task light system for a surgical console includes a task light that can be held to a coupling location on a surgical console. The task light has an inductive coupling, a light source, and a goose neck connecting the light source to the inductive coupling. The coupling location is located on a face of the surgical console. The coupling location is configured to magnetically attract and hold the inductive coupling. The face of the surgical console where the coupling location is located is a continuous surface.

### BACKGROUND OF THE INVENTION

The present invention relates to an inductive task light that is magnetically coupled to a surface of a surgical console.

Most ophthalmic surgeries today are performed with the use of complex surgical consoles in darkened operating rooms. During an operation, a surgical microscope and special light source are used to illuminate and view the eye. In most cases, the overhead light in the operating room is turned off during surgery. Unlike operations performed on other parts of the body (which often require very bright illumination), operations performed on the eye require specialized illumination by lighting that is precisely controlled. In some cases, there is no general illumination of the operating room.

In this darkened environment, it is sometimes difficult for an assisting nurse to see the various instruments, implantable devices, disposables, etc. that are used during an operation. Typically, a nurse uses a separate light source that can be focused on the tray that holds these items. In some cases, this separate light source is a standalone lamp that is plugged into the wall. In other cases, a battery powered lamp may be used. The location of these lamps is often fixed which limits their usefulness. Moreover, these lamps have separate sockets or plugs that do not allow for a clean surface.

It would be desirable to have a specialized task light that allows for the precise placement of light on a work area, such as a tray. It would also be desirable to have a task light that does not have cords or plugs.

### SUMMARY OF THE INVENTION

In one embodiment consistent with the principles of the present invention, the present invention is a task light system for a surgical console. The task light system includes a task light that can be held to a coupling location on a surgical console. The task light has an inductive coupling, a light source, and a goose neck connecting the light source to the inductive coupling. The coupling location is located on a face of the surgical console. The coupling location is configured to magnetically attract and hold the inductive coupling. The face of the surgical console where the coupling location is located is a continuous surface.

In another embodiment consistent with the principles of the present invention, the present invention is a task light for a surgical console. The task light has an inductive coupling, a light source, and a goose neck connecting the light source to the inductive coupling. The inductive coupling is magnetically attracted to one or more coupling locations located on a face of a surgical console. The one or more coupling locations are configured to hold the inductive coupling.

It is to be understood that both the foregoing general description and the following detailed description are exemplary and explanatory only and are intended to provide further explanation of the invention as claimed. The following description, as well as the practice of the invention, set forth and suggest additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of a surgical console with one or more receptacle areas for an inductive task light according to the principles of the present invention.

FIG. 2 is a side view of an inductive task light according to the principles of the present invention.

FIG. 3 is a cut away view of a coupling section of an inductive task light according to the principles of the present invention.

FIG. 4 is a cut away view of a coupling section of surgical console according to the principles of the present invention.

FIG. 5 is a cut away view of the interfacing of a coupling section of an inductive task light and a coupling section of a surgical console according to the principles of the present invention.

### DETAILED DESCRIPTION OF THE PREFERRED EMBODIMENTS

Reference is now made in detail to the exemplary embodiments of the invention, examples of which are illustrated in the accompanying drawings.

Wherever possible, the same reference numbers are used throughout the drawings to refer to the same or like parts.

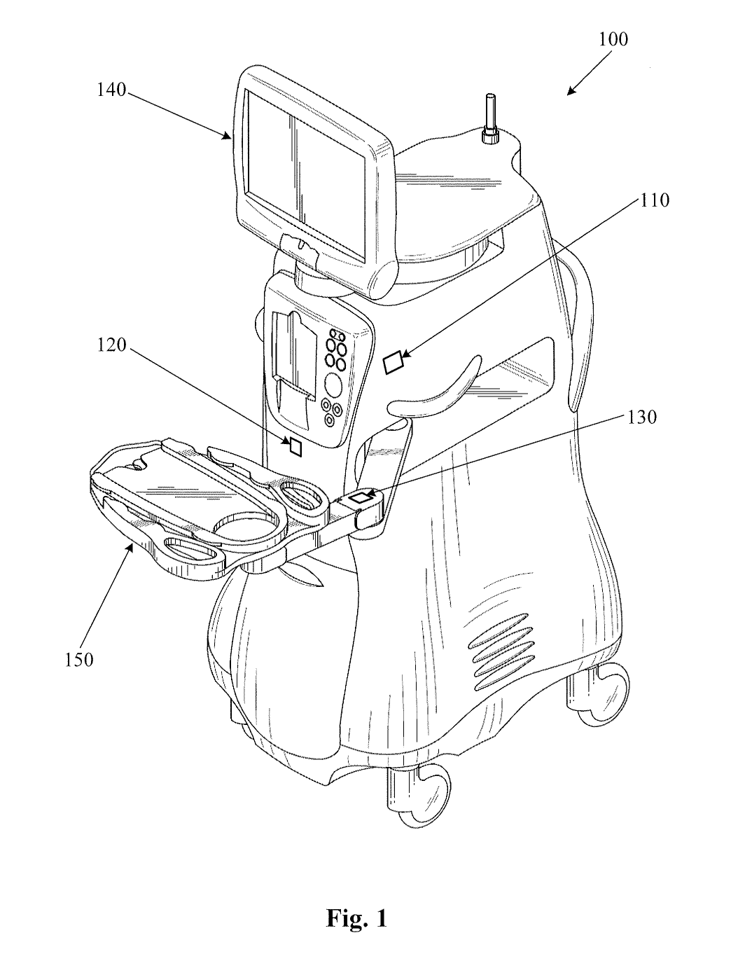

FIG. 1 is a perspective view of a surgical console with one or more receptacle areas for an inductive task light according to the principles of the present invention. Surgical console 100 is typical of surgical consoles commonly used for ophthalmic surgery. Surgical console 100 has a touch screen 140 that controls the operation of the console and a tray 150 for holding the various instruments, implantable devices, disposables, etc. that are used during surgery.

In FIG. 1, surgical console has three locations (110, 120, and 130) that are designed to couple with an inductive task light as more fully described below. Each of these three coupling locations (110, 120, and 130) is flush with the surface on which it is located. In other words, the surface that includes these coupling locations (110, 120, and 130) is continuous so as to provide a surface that can be cleaned easily. There are no crevices or the like on these surfaces that can collect dirt and germs. Coupling location 110 is located on a side of the surgical console 100 near the front face (where the tray 150 and touch screen 140 are located). Coupling location 120 is located on the front of the surgical console 100. Coupling location 130 is located on the arm that holds tray 150. While three coupling locations (110, 120, and 130) are depicted in FIG. 1, any number of coupling locations may be present. In one embodiment of the present invention, only one coupling location is present. Moreover, the coupling location(s) may be located anywhere on the surgical console 100 or the tray 150. The exemplary coupling locations (110, 120, and 130) of FIG. 1 are located so as to provide a good location for the task light.

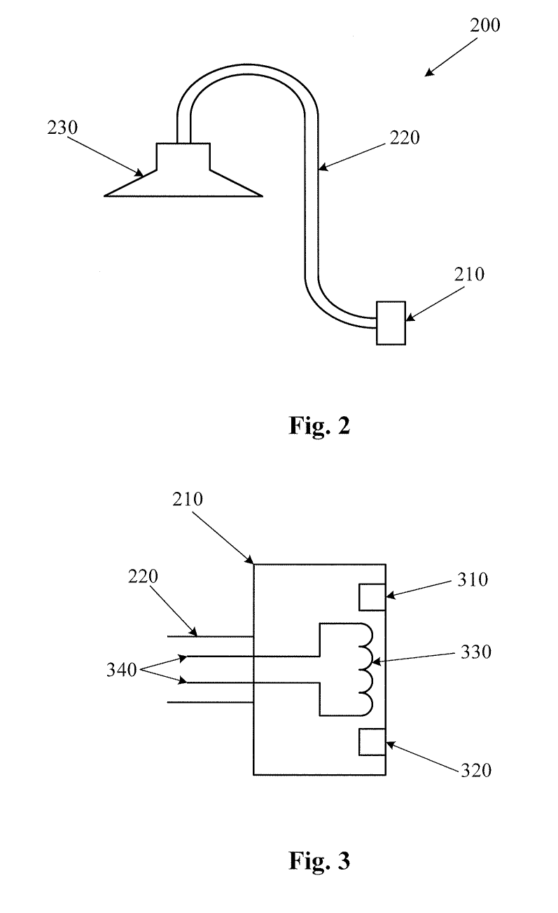

FIG. 2 is a side view of an inductive task light according to the principles of the present invention. In FIG. 2, task light 200 has an indicative coupling 210, a flexible goose neck 220, and a lamp head 230. The inductive coupling 210 is configured to interface with the coupling locations (110, 120, and 130). In this manner power can be provided to the task light 200. The flexible goose neck 220 allows for the precise placement of lamp head 230.

Lamp head 230 is designed to cast light on a specific area, such as tray 150. Lamp head 230 contains a light source such as an incandescent bulb, fluorescent bulb, or light emitting diodes (LEDs). In one embodiment of the present invention, an array of LEDs is housed in lamp head 230. The type of LEDs selected produce a white light that suitable for viewing tray 150. Optionally, a filter can be used to filter out wavelengths that can be harmful to the eye. In this manner, lamp head 230 can emit a light that is similar in quality to that used to illuminate the eye during surgery. It is known that certain wavelengths of light can damage the retina. These wavelengths can be filtered out of the light produced by lamp head 230.

FIG. 3 is a cut away view of a coupling section of an inductive task light according to the principles of the present invention. In FIG. 3, inductive coupling 210 includes magnets 310 and 320, coil 330, and wires 340. Wires 340 are located in flexible goose neck 220 and conduct current to the light source in lamp head 230. Magnets 310 and 320 are attracted to similar magnets at a coupling location as described below. Coil 330 inductively couples to a similar coil at a coupling location as described below.

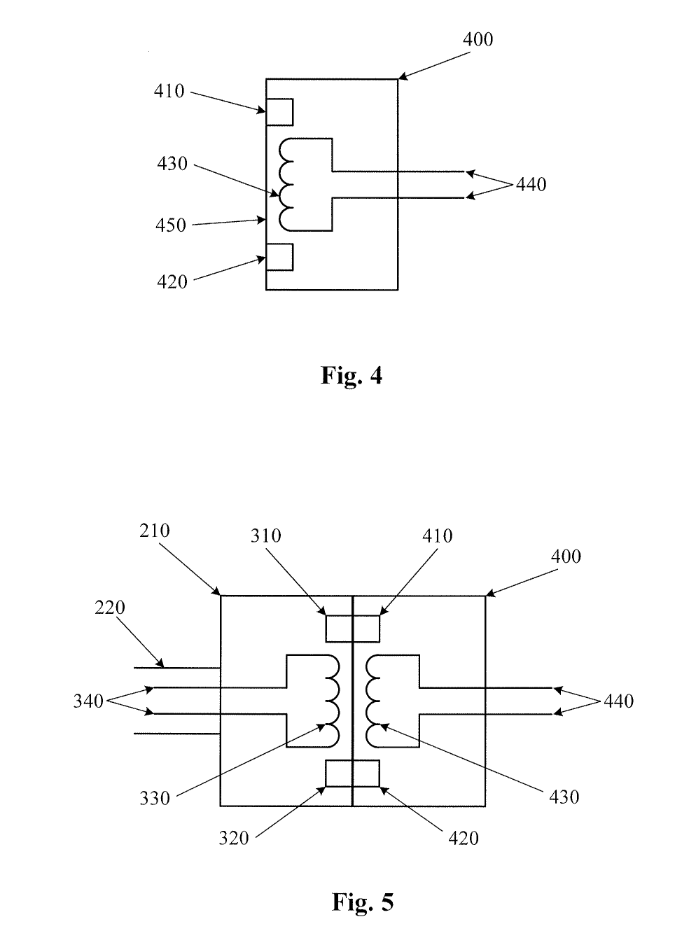

FIG. 4 is a cut away view of a coupling section of surgical console according to the principles of the present invention. In FIG. 4, coupling location 400 includes magnets 410 and 420, coil 430, and wires 440. Wires 440 are located in surgical console 100 provide a source of power to the light source in lamp head 230. Magnets 410 and 420 are attracted to similar magnets located in an inductive coupling as described below. Coil 430 inductively couples to a similar coil located in an inductive coupling as described below.

Magnets 410 and 420 are located behind the face 450 of a surface of surgical console 100. In one embodiment of the present invention, magnets 410 and 420 are located behind the outer skin of surgical console 100. The outer skin of surgical console 100 can be continuous (without crevices) where magnets 410 and 420 are located. In addition, the outer skin can have a drawing on its face that indicates the location of coupling location 400. For example, a square or a circle can be drawn on the face of surgical console 100 at the coupling location 400.

FIG. 5 is a cut away view of the interfacing of a coupling section of an inductive task light and a coupling section of a surgical console according to the principles of the present invention. FIG. 5 shows how inductive coupling 210 interfaces with coupling location 400. In FIG. 5, magnets 310 and 320 of inductive coupling 210 are attracted to magnets 410 and 420 of coupling location 400. This magnetic attraction secures inductive coupling 210 to coupling location 400 (thereby securing task light 200 to surgical console 100). In addition, this magnetic attraction serves to align coils 330 and 430. In other words, inductive coupling 210 sticks to the surface of surgical console 100 at coupling location 400 and is held there by magnets 310, 320, 410, and 420. While two magnets are shown, any number of magnets may be used to properly attach inductive coupling 210 to coupling location 400 and to properly orient coils 330 and 430.

When secured to coupling location 400, inductive coupling 210 is located such that coils 330 and 430 are inductively coupled. In this manner, coil 430 acts like the primary coil of a transformer, and coil 330 acts like the secondary coil of a transformer. When a current is passed through coil 430, a magnetic filed is produced. This magnetic field couples with coil 330 and induces a current in it. This current is carried by wires 340 to the light source of task light 200.

In one embodiment of the present invention, task light 200 has a motion sensor that activates the light source. The motion sensor acts to turn on task light 200 when it senses motion. In this manner, task light 200 can be turned on via the motion sensor, stay on for a preset period of time, and then automatically turn off This allows for hands free operation of task light 200 by a nurse in the operating room.

In another embodiment of the present invention, the operation of task light 200 is controlled by the touch screen 140 of surgical console 100. In this manner, the touch screen 140 allows task light 200 to be turned on and off and for the intensity of task light 200 to be adjusted. In one embodiment of the present invention, virtual buttons located on touch screen 140 simulate an on/off switch and a dimmer switch.

In another embodiment of the present invention, the task light 200 has an attached magnifying glass (not shown) so that the nurse can read small lettering on surgical packaging, surgical implements, surgical devices (like intraocular lenses), and the like.

From the above, it may be appreciated that the present invention provides an inductive task light that provides targeted illumination in a darkened operating room environment. The present invention provides a task light that is magnetically coupled to the surface of a surgical console. The present invention is illustrated herein by example, and various modifications may be made by a person of ordinary skill in the art.

Other embodiments of the invention will be apparent to those skilled in the art from consideration of the specification and practice of the invention disclosed herein.

It is intended that the specification and examples be considered as exemplary only, with a true scope and spirit of the invention being indicated by the following claims.

What is claimed is:

1. A task light system comprising:
a task light comprising an inductive coupling, a light source, and a goose neck connecting the light source to the inductive coupling; and
a coupling location located on a face of a surgical console, the coupling location configured to magnetically attract and hold the inductive coupling to the coupling location, wherein the face of the surgical console where the coupling location is located is a continuous surface.

2. The task light system of claim 1 wherein the inductive coupling comprises:
a magnet,
a coil, and
a housing at least partially enclosing the magnet and the coil.

3. The task light system of claim 1 wherein the light source comprises a light emitting diode.

4. The task light system of claim 1 wherein the goose neck allows for the light source to be variably placed.

5. The task light system of claim 1 further comprising:
a second coupling location located on a face of a surgical console, the second coupling location configured to magnetically attract and hold the inductive coupling to the second coupling location, wherein the face of the surgical console where the second coupling location is located is a continuous surface.

6. The task light system of claim 1 wherein the coupling location comprises:
a magnet,
a coil, and
a section of the face of the surgical console behind which the magnet and the coil are located, the section of the face of the surgical machine being continuous and without crevices.

7. The task light system of claim 1 wherein the inductive coupling is attachable to the coupling location, and when attached, the inductive coupling is magnetically coupled to the coupling location.

8. The task light system of claim 1 wherein the task light further comprises:
a motion sensor, the motion sensor for turning the light source on and off.

9. The task light system of claim 1 wherein the task light is controllable from a touch screen on the surgical console.

10. The task light system of claim 2 wherein a current is induced in the coil when the inductive coupling is held to the coupling location.

11. A task light for a surgical console, the task light comprising:
an inductive coupling,
a light source, and
a goose neck connecting the light source to the inductive coupling; and
wherein the inductive coupling is magnetically attracted to one or more coupling locations located on a face of a surgical console, the one or more coupling locations configured to hold the inductive coupling to the one or more coupling locations.

12. The task light of claim 11 wherein the inductive coupling comprises:
a magnet,
a coil, and
a housing at least partially enclosing the magnet and the coil.

13. The task light of claim 11 wherein the light source comprises a light emitting diode.

14. The task light of claim 11 wherein the goose neck allows for the light source to be variably placed.

15. The task light of claim 11 wherein the inductive coupling is attachable to the coupling location, and when attached, the inductive coupling is magnetically coupled to the coupling location.

16. The task light of claim 11 further comprising:
a motion sensor, the motion sensor for turning the light source on and off

17. The task light of claim 1 wherein the task light is controllable from a touch screen on the surgical console.

18. The task light of claim 12 wherein a current is induced in the coil when the inductive coupling is held to the coupling location.
